(19) United States
(12) Patent Application Publication
(10) Pub. No.: US 2016/0331518 A1
(43) Pub. Date: Nov. 17, 2016

(54) HIGH-VISCOSITY FILLING OF IMPLANTED DEVICES

(71) Applicants: Craig Alan CABLE II, Mission Viejo, CA (US); Charles DEBOER, Sierra Madre, CA (US); Mark S HUMAYUN, Glendale, CA (US); Yu-Chong TAI, Pasadena, DC (US); Sean CAFFEY, Pasadena, CA (US)

(72) Inventors: Craig Alan CABLE II, Mission Viejo, CA (US); Charles DEBOER, Sierra Madre, CA (US); Mark S HUMAYUN, Glendale, CA (US); Yu-Chong TAI, Pasadena, DC (US); Sean CAFFEY, Pasadena, CA (US)

(21) Appl. No.: 15/137,234

(22) Filed: Apr. 25, 2016

Related U.S. Application Data
(60) Provisional application No. 62/159,689, filed on May 11, 2015.

(51) Int. Cl. A61F 2/16

(52) U.S. Cl. CPC A61F 2/16; A61F 2/12

(57) ABSTRACT

In various embodiments, high-viscosity fluids are utilized to fill implantable reservoirs while filling accuracy is improved via elimination of dead space and/or utilization of pushing fluids to urge the high-viscosity fluid into the implantable reservoir.

### RELATED APPLICATION

This application claims the benefit of and priority to U.S. Provisional Patent Application No. 62/159,689, filed May 11, 2015, the entire disclosure of which is hereby incorporated herein by reference.

### TECHNICAL FIELD

In various embodiments, the present invention relates generally to tools used to fill an implanted device, such as a fluid-filled intraocular lens.

### BACKGROUND

Fluid-filled implantable reservoirs (FFIRs) are used in numerous medical applications and at various anatomic sites. Such reservoirs may, for example, serve as intraocular lenses (IOLs); may be used to position tissue and/or provide tissue compression or spacing during surgery; and may deliver pharmaceuticals.

IOLs are used to replace the natural crystalline lens after cataract removal. First the cataract is surgically removed from the capsule of the crystalline lens. To implant an IOL, an incision is made in the cornea, followed by a capsulorhexis—i.e., removal of a portion of the lens capsule to provide surgical access to the natural lens, which is itself removed using phacoemulsification to fragment and aspirate the lens from the lens capsule. The IOL, in a folded conformation, is then injected into the lens capsule. The implanted IOL is filled with liquid to provide the appropriate amount of vision correction. A liquid-filled IOL that provides accommodation—made from, for example, an elastic, biocompatible polymer—results in numerous benefits and advantages, e.g., the ability to adjust the lens following implantation; to customize the lens to the needs of each patient; to accommodate vision; sharper vision over a wide range of distances; and reduction of visual side effects such as glares and halos. See, e.g., U.S. Pat. No. 8,771,347, and U.S. patent application Ser. No. 13/473,012, filed May 16, 2012, the entire disclosure of each of which is hereby incorporated by reference.

Insertion of an IOL in an unfilled state offers significant clinical advantages, stemming primarily from the small capsulorhexis diameter required for IOL introduction. This reduces post-operative healing times, allows the surgeon to avoid use of sutures for closing the incision, and reduces post-operative astigmatism. Incisions less than 3 mm, and preferably less than 2 mm, are desired by operating personnel for better surgical outcomes. In addition, the optical properties of certain liquid-filled IOLs can be adjusted after implantation to ensure accurate vision by refractive corrections; this is achieved through adjustment of the filling medium inside the lens. When made flexible, IOLs can provide adjustable focal distances (i.e., accommodation), relying on the natural focusing ability of the eye (e.g., using contractions of ciliary muscles). These IOLs are often filled with a high-viscosity fluid, which tends to be more stable than a low-viscosity fluid. In addition, filling preferably occurs with few or no air bubbles, which can cause unwanted visual disturbances and overinflate the lens. Filling accuracy is critical for proper visual performance and, due to the small size of the reservoir, filling precision is essential nominally under 30 μL, and ideally within 2 μL.

Similar considerations attend the use of other implantable reservoirs, since implanted devices are necessarily space limited and desirably are introduced using minimally invasive surgery. Air bubbles can cause undesired effects even if the reservoir does not perform an optical function; such effects may include reservoir damage, or unwanted intracorporeal injection or diffusion of air. Moreover, the delivery fluid (e.g., biologicals such as monoclonal antibodies, chemotherapeutics, RNAi therapeutics, etc.) may well be expensive and its waste therefore costly. Accordingly, there is a need to administer no more fluid than is necessary, and without waste, when filling a liquid-filled implantable reservoir.

### SUMMARY

Embodiments of the present invention include a system for filling an implant reservoir with high-viscosity fluid. (As used herein, the term “fluid” generally refers to a liquid or a mixture of liquids which, depending on the context, may or may not include one or more gases dissolved and/or dispersed therein. In general, the term fluid does not include pure gases.) Exemplary reservoirs include but are not limited to fluid-filled IOLs, breast implants, drug reservoirs, inflatable balloons for moving tissue planes, and inflatable scleral buckles. In some configurations, the system may be used in conjunction with the above-mentioned reservoir types to fill to a specified amount, purge or remove fluid, exchange fluid, and/or prime to a certain level (e.g., percentage of air, old fluid, or concentration of a combination of fluids). In various embodiments, the system includes or consists essentially of a fluid reservoir, a pump, and an implantable reservoir. The implantable reservoir may be either an implanted device with a fluid reservoir or a device that has a fluid reservoir that is in contact with the patient (e.g., an external refillable reservoir). In accordance with various embodiments, the system includes a processing module that receives patient parameters and signals from sensors and/or the implantable device. The signals may be automatically sent to (or requested and obtained by) the processing module, or manually entered into the processing module. Based on the received signals, the processing module may control the pump directly or direct a human operator to control the pump appropriately. The fluid system may have multiple sensors, filters, or permeable/semipermeable membranes placed along the fluid pathway. Embodiments of the invention may include one or more pumps to provide dispensing, aspiration, and vacuum.

Before filling occurs, a vacuum pump may pull a vacuum to evacuate air from one or more residual air spaces (i.e., dead volume) in the system, such as in the fluidic connection lines, fitting, and/or the implantable reservoir. When this occurs, the volume of the dead space may remain the same; however, the total number of air molecules in the dead space decreases. This may be explained using the ideal gas law:

PV=nRT

where P is pressure, V is volume, n is the number of molecules of the gas, R is the ideal gas constant, and T is the temperature of the gas. When the pressure is 1 atmosphere there are n molecules of air. If the air is evacuated or removed due to application of a vacuum level of 10⁻⁴ atmosphere, the result is n×10⁻⁴ molecules of air. If the volume were returned to atmospheric pressure by allowing it to contract, the air in the new dead volume within the system would be reduced by a factor of 10⁻⁴. (For example, 1 microliter of dead-volume air would become 10⁻⁴ microliter of dead-volume air.) Thus, the effective dead volume may be reduced in this manner. In accordance with embodiments of the invention, dead volume may be reduced by holding the infusion liquid at a specific point in the line and pulling vacuum to the air portion of the line. This results in an isovolumetric condition in which air molecules are removed. Then, upon injecting the fluid, a much smaller amount of air is infused. In certain embodiments, the injected volume of air may have approximately zero volume, depending on the level of vacuum applied to the dead space.

The technique described above may be applied to collapsible as well as non-collapsing reservoirs. For example, a non-collapsible reservoir may have a fixed volume that is filled with air. A vacuum may then be applied to the reservoir, thereby removing most or essentially all of the air. Fluid may then be used to fill the evacuated reservoir, leaving substantially no air in the reservoir. Likewise, a collapsible reservoir would collapse when subjected to vacuum, allowing the air to be evacuated before filling. Thus, techniques in accordance with embodiments of the invention substantially eliminate or minimize the air in a dead volume area, which would otherwise subsequently be either remnant in the reservoir or injected into the implantable reservoir.

Various embodiments of the invention may be sterilized for use in an implant. For example, the reservoir and injection system may be sterilized and packaged. The reservoir may then connect to the injection system using a sterile connection while also having the fluid within sterilized. Such systems may have either a pre-accessed reservoir or a reservoir that is easy to access and load for injection. Systems may also allow an operator to load the front of the fluidic line with the fluid to be injected into the implantable reservoir. A second liquid may then be primed behind the implantable liquid. A membrane or barrier may be placed between the two fluids, in various embodiments, to maintain sterility. The second fluid may then push the implantable liquid into the implantable reservoir. Such embodiments may decrease the amount of implantable fluid needed, as such liquids (e.g., injectable drugs) may be expensive. Portions of the implantable fluid may be replaced with an inexpensive fluid present to occupy otherwise dead volume (i.e., displace air therefrom) and not, at least in appreciable volumes, be injected into the reservoir.

Various embodiments of the invention involve filling with high-viscosity fluids. For example, filling techniques in accordance with embodiments of the invention include accounting for line and air bubble compliance during filling, and using non-linear or stepped filling techniques and/or wait times before removing the filling line from the implantable reservoir. Moreover, embodiments of the invention relate to precise filling for optimizing the optical aberration of an IOL.

Embodiments of the invention include different configurations, including full systems including a main system and replaceable disposable fluidics, a handheld system, a combination of many functional units, or a combination thereof.

In various embodiments, a handheld system is utilized for insertion and/or filling the implantable reservoir with high-viscosity fluid. Exemplary reservoirs include but are not limited to fluid filled intraocular lenses, breast implants, drug reservoirs, inflatable balloons for moving tissue planes, and inflatable scleral buckles. In various embodiments, the system may be used to fill to a specified amount, purge or remove fluid, exchange fluid, or prime to a certain fluid level (e.g., a percentage of air, old fluid, or concentration of a combination of fluids). Systems may include, consist essentially of, or consist of one or more fluid reservoirs, an actuation mechanism to move the fluid (e.g., a pump or plunger), and a fluid line fluidly that may be fluidly coupled to the implantable reservoir. The implantable reservoir may be, for example, an implanted device with a fluid reservoir or a device having a fluid reservoir in contact with the patient (e.g., an external refillable reservoir). The actuation of the fluid may be accomplished by a pump, pressure differentials, mechanical springs and/or tensioners, or manual actuation.

In various embodiments, the fluid reservoir is a prefilled reservoir that is specifically marked for certain fills or operations (such as priming, degassing, deployment, etc.) Such prefilled reservoirs may be cartridges that fluidly couple to a hand piece or come preassembled with the hand piece. A cartridge may be a fluid reservoir that comes into fluid connection with the hand piece and that is self-actuated, actuated by an exterior mechanism (e.g., a pump), or is manually actuated once connected to the hand piece.

Advantageously, implantable reservoirs in accordance with embodiments of the present invention may be at least partially filled after implantation within a patient and/or have their fill levels adjusted (e.g., via removal or addition of fluid) while still implanted without the need for extraction and/or replacement. In various embodiments, the implantable reservoir may be partially filled prior to implantation and partially filled after implantation.

In an aspect, embodiments of the invention feature a system for filling an implantable reservoir with a high-viscosity fluid. The system includes or consists essentially of a first fluid line for fluidly coupling to the implantable reservoir, a second fluid line for conducting the high-viscosity fluid to the first fluid line, a first pump fluidly coupled to the first and second fluid lines, a first fluid reservoir for containing the high-viscosity fluid, and a second pump for pumping high-viscosity fluid from the first fluid reservoir to the implantable reservoir via the first fluid line. The second fluid line is fluidly coupled to the first fluid line. The first pump evacuates air (and/or other gas) from at least the first fluid line (and in some embodiments, also the second fluid line), thereby eliminating dead space therefrom. The first fluid reservoir is fluidly coupled to the second fluid line.

Embodiments of the invention may include one or more of the following in any of a variety of combinations. The high-viscosity fluid may include, consist essentially of, or consist of one or more liquids. The high-viscosity fluid may have a viscosity of at least 100 centipose. The high-viscosity fluid may have a viscosity of at least 1000 centipose. At least portions of the first and second fluid lines may be concentric. The second fluid line may terminate within the first fluid line upstream of a terminus thereof. The terminus of the first fluid line may be coupled to the implantable reservoir. The system may include a control system for controlling the first pump and/or the second pump. The system may include one or more sensors for measuring flow rate and/or pressure within the first fluid line and/or the second fluid line. The control system may be responsive to signals received from the one or more sensors. The control system may control the second pump based at least in part on compliance within the system (e.g., compliance sensed by one or more sensors such as optical sensors, pressure sensors, flow-rate sensors, etc.) The system may include one or more heating mechanisms (e.g., heaters and/or heating elements) disposed along and/or within at least a portion of the first fluid line, the second fluid line, and/or the first fluid reservoir. The implantable reservoir may include, consist essentially of, or consist of an intraocular lens. The control system may control the flow of the high-viscosity fluid into the implantable reservoir based on one or more patient parameters. The one or more patient parameters may include, consist essentially of, or consist of one or more of lens capsule geometry, lens size, lens position in the eye, patient age, corneal shape, lens refractive index, desired optical power, accommodation (e.g., determined at least in part by one or more other patient parameters), and/or nominal fill of the intraocular lens (e.g., determined at least in part by one or more other patient parameters). The system may include a valve configured to fluidly couple the first fluid line to the implantable reservoir and fluidly uncouple the first fluid line from the implantable reservoir. One or more semipermeable membranes may be disposed within the second fluid line. The one or more semipermeable membranes may allow flow of gas therethrough without allowing flow of liquid therethrough. The system may include, fluidly coupled to the second fluid line, a second fluid reservoir for containing a pushing fluid for exerting force on the high-viscosity fluid. The second fluid reservoir may include a pushing fluid different from the high-viscosity fluid. The pushing fluid may include, consist essentially of, or consist of one or more liquids. The system may be a handheld system.

In another aspect, embodiments of the invention feature a method of filing an implantable reservoir with high-viscosity fluid though a first fluid line fluidly coupled to the implantable reservoir. The high-viscosity fluid is introduced into a second fluid line fluidly coupled to the first fluid line. Air (and/or one or more other gases) is evacuated from the first fluid line. Thereafter, the high-viscosity fluid is urged into the implantable reservoir via the first fluid line.

Embodiments of the invention may include one or more of the following in any of a variety of combinations. The high-viscosity fluid may include, consist essentially of, or consist of one or more liquids. At least portions of the first and second fluid lines may be concentric. The second fluid line may terminate within the first fluid line upstream of a terminus thereof. The terminus of the first fluid line may be coupled to the implantable reservoir. The high-viscosity fluid may be introduced into the second fluid line via a third fluid line fluidly coupled to the first and second fluid lines. Before urging the high-viscosity fluid into the implantable reservoir, air (and/or one or more other gases) may be evacuated from the implantable reservoir via the first fluid line. The high-viscosity fluid may be urged into the implantable reservoir by a pushing fluid, different from the high-viscosity fluid, disposed within the second fluid line. The pushing fluid may include, consist essentially of, or consist of one or more liquids. Air (and/or one or more other gases) may be evacuated from at least a portion of the second fluid line prior to introducing the high-viscosity fluid into the second fluid line.

In yet another aspect, embodiments of the invention feature a method of filing an implantable reservoir with a pre-determined amount of high-viscosity fluid though a fluid line fluidly coupled to the implantable reservoir. The pre-determined amount of high-viscosity fluid is disposed into a first region of the fluid line, and a pushing fluid is disposed into a second region of the fluid line. The pushing fluid is different from the high-viscosity fluid. The first region of the fluid line is disposed upstream of the implantable reservoir and downstream of the second region of the fluid line. Force is applied to the pushing fluid to thereby urge the high-viscosity fluid into the implantable reservoir. Substantially no pushing fluid may enter the implantable reservoir.

Embodiments of the invention may include one or more of the following in any of a variety of combinations. The high-viscosity fluid may include, consist essentially of, or consist of one or more liquids. The pushing fluid may include, consist essentially of, or consist of one or more liquids. The high-viscosity fluid and the pushing fluid may be immiscible and in contact with each other within the fluid line. A moveable mechanical boundary may be disposed between the pushing fluid and the high-viscosity fluid. Movement of the mechanical boundary may urge the high-viscosity fluid into the implantable reservoir. Air (and/or one or more other gases) may be evacuated from the first region of the fluid line before disposing the high-viscosity fluid therein.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic diagram of a system for high-viscosity filling of implantable reservoirs in accordance with embodiments of the invention;

FIG. 2 is a graph depicting an exemplary half-life dependence of compliance volume with time in accordance with embodiments of the invention;

FIGS. 3A-3C are schematic cross-sections of portions of systems for filling implantable reservoirs while minimizing dead space in accordance with embodiments of the invention;

FIG. 4 is a graph of exemplary defocus curves for three different implantable lenses in accordance with embodiments of the invention;

FIGS. 5A and 5B are schematic cross-sections of portions of systems for filling implantable reservoirs while minimizing dead space in accordance with embodiments of the invention;

FIGS. 6A-6D are schematic cross-sections of portions of systems for filling implantable reservoirs in which a second fluid is utilized to urge filling fluid toward the reservoir in accordance with embodiments of the invention;

FIG. 7 is a schematic diagram of a system for high-viscosity filling of implantable reservoirs in accordance with embodiments of the invention; and

FIG. 8 is a schematic cross-section of a hand-held system for high-viscosity filling of implantable reservoirs in accordance with embodiments of the invention.

### DETAILED DESCRIPTION

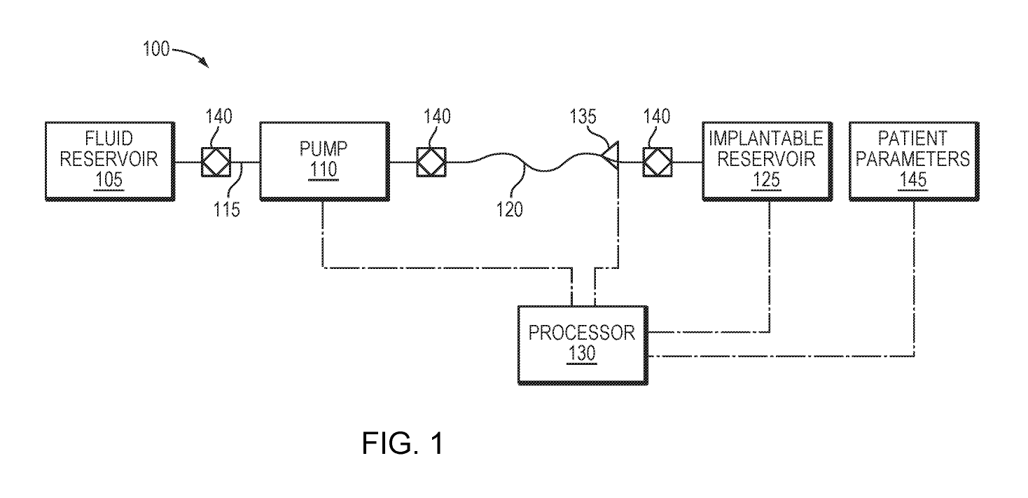

FIG. 1 is a schematic diagram for an exemplary filling and aspiration system 100 for a high-viscosity fluid in accordance with embodiments of the present invention. (As utilized herein, the term “high-viscosity” refers to a viscosity of at least 10 centipoise; in various embodiments, high-viscosity fluids may have viscosities of at least 100 centipoise, or even at least 1000 centipoise.) As shown in FIG. 1, system 100 includes a high-viscosity fluid reservoir 105 that is fluidly coupled to a pump 110 by a fluidic line 115. The high-viscosity fluid then flows from the pump 110 through another fluidic line 120 to an implantable reservoir 125. The pump 110 is controlled either directly by a controller 130, or the controller 130 may direct a human operator to control the pump 110.

The fluidic lines 115, 120 may experience higher pressures during filling due to the high-viscosity fluid within the lines. As known to those of skill in the art, pressure drop through a line is linearly related to the viscosity, as demonstrated via the Hagen-Poiseuille equation,

$$Δ P = \frac{8 μ LQ}{π {r}^{2}} ,$$

where ΔP is the pressure drop across the line, μ is the dynamic viscosity, L is the length of the line, Q is the volumetric flow rate, and r is the inner radius of the line. Thus, in accordance with embodiments of the invention, system 100 is usable and utilized at high operating pressures, e.g., pressures of at least 10 psi. In various embodiments, the operating pressure may be between 20 psi and 10,000 psi, between 20 psi and 1000 psi, or between 50 psi and 500 psi. Such high pressures may cause problems in metering fluid flow, as pump reading and many inline sensing mechanisms may become compromised, thereby reducing the accuracy of fluid filling. In various embodiments, the compliance from high-pressure, high-viscosity fluid systems arises at least in part from the walls of the fluidic lines having compliance and air bubbles within the fluid being compressed. As utilized herein, the term “compliance” refers to component expansion that results in additional effective volume within a system. Fluidic lines may cause compliance by expanding under high pressure. As the internal pressure within the line increases, the inner walls of the fluidic lines 115, 120 expand. This expansion changes the volume within the fluidic line 120, meaning that fluid that is meant to be flowing from the pump 110 to the implantable reservoir 125 is instead being used to fill the expanding volume of the fluidic line 120. Thus, not all of the fluid coming from the pump 110 is going to the implantable reservoir 125 (i.e., some of the fluid is used to fill the expanding volume of the fluidic line 120).

Air bubbles of any size may cause the same compliance problems. Air bubbles are compressible (unlike most high-viscous fluids, which are generally considered to be incompressible). Since the mass of the air and the temperature within the viscous fluid may be assumed to remain the same, Boyle's Law states that P₁V₁=P₂V₂, where P is pressure and V is volume of the air bubble. The subscripts denote different states of the air bubble. In state 1 the fluid is assumed to be at atmospheric pressure. When the pump 110 starts to push high-viscosity fluid through the fluidic line 120, the pressure increases to state 2. Pursuant to Boyle's Law, the air bubble decreases in volume as high-viscosity fluid starts to flow through the fluidic line 120. Thus, as fluid is pumped from the pump 110 to the implantable device 125, the volume occupied by air decreases. This volume decrease is offset as the fluid fills the extra space. Thus, not all of the fluid flowing out of the pump 102 may reach the implantable device 125. The following formula may be used to represent the system filling:

Vₚₘₚ(t)=Vᵢₘₚₗₐₙₜ(t)+V_{c}(t),

where Vₚₘₚ is the volume displaced by the pump 110 as a function of time, Vᵢₘₚₗₐₙₜ is the volume of fluid displaced in the implantable reservoir 125 as a function of time, and V_{c} is the amount of volume change due to compliance in the system as a function of time. Each volume is a function of time due to the volume differences at different times while the system is operating. Thus, in various embodiments of the invention, sensors 135 and air bubble removal or monitoring devices 140 are utilized in various locations in system 100 (e.g., along the fluidic lines 115, 120). Such devices may serve as an extra meter for measuring the amount of fluid flowing though the fluidic lines 115, 120. An air monitoring sensor may also be placed along the fluidic lines 115, 120 to estimate the amount of air traveling in the lines, either during infusion or aspiration. Sensors usable in embodiments of the present invention include flow meters, pressure meters, strain gauges, and velocimeters. Sensing may occur directly in line with the flow, or may be completed remotely. For example, ultrasound sensing may be used outside fluidic lines 115, 120 and the output thereof may be utilized (e.g., by controller 130) to determine the amount of fluid that has been dispensed into or aspirated from the implantable reservoir 125. Instead or in addition, the implantable reservoir 125 may have one or more sensors that provide indications relevant to the operation of system 100 to controller 130 and/or to a human operator. Such indications may include measurements of current fill amount, flow rate, line pressure, line compliance, amount of air in the lines, and/or amount of air and/or fluid that has passed into the implantable reservoir 125. The indications may be relayed via, for example, gauges (e.g., dials), lights, series of lights (e.g., light bars), and/or by audible alerts. Patient-specific parameters 145 may also be used as a feedback signal to the controller 130. The patient parameters 145 are, in various embodiments, physical parameters that may be measured from the patient that provide an indication regarding the fill status of the implantable reservoir 125. In various embodiments, patient parameters 145 may include or consist essentially of measurements acquired from the patient or from a device within the patient.

The fluid reservoir 105, fluidic lines, and any part of the fluidic path may further include a heating mechanism (e.g., a heater and/or heating element) to alter the viscosity of the high-viscosity fluid to reduce the effect of the difficulties arising from higher viscosity. The heating mechanism may be responsive to the controller 130 (e.g., to signals received from the controller). As known to those skilled in the art, viscosity typically decreases with increasing temperature (up to the critical temperature which is the highest temperature at which a substance can exist in liquid form regardless of pressure), as demonstrated via the Arrhenius-type equation,

$$μ = {μ}_{0} {e}^{\frac{E μ}{RT}} ,$$

where E_{μ} is the temperature coefficient of viscosity. Thus, temperature changes to the high-viscosity fluid may decrease the required pumping operating pressure, as well as possible errors in fill volume caused by compliance.

The controller (or “processor”) 130 (and/or any or all of its components) may include or consist essentially of a general-purpose microprocessor, but depending on implementation may alternatively be a microcontroller, peripheral integrated circuit element, a customer-specific integrated circuit (CSIC), an application-specific integrated circuit (ASIC), a logic circuit, a digital signal processor, a programmable logic device such as a field-programmable gate array (FPGA), a programmable logic device (PLD), a programmable logic array (PLA), an RFID processor, smart chip, or any other device or arrangement of devices that is capable of implementing the steps of the processes of embodiments of the invention. Moreover, at least some of the functions of controller 130 may be implemented in software and/or as mixed hardware-software modules. Software programs implementing the functionality herein described may be written in any of a number of high level languages such as FORTRAN, PASCAL, JAVA, C, C++, C#, BASIC, various scripting languages, and/or HTML. Additionally, the software may be implemented in an assembly language directed to a microprocessor resident in controller 130. The software may be embodied on an article of manufacture including, but not limited to, a floppy disk, a jump drive, a hard disk, an optical disk, a magnetic tape, a PROM, an EPROM, EEPROM, field-programmable gate array, CDROM, or DVDROM. Embodiments using hardware-software modules may be implemented using, for example, one or more FPGA, CPLD, or ASIC processors.

Various embodiments of the present invention may involve the filling of a fluid-filled IOL. In an exemplary embodiment, the filling fluid includes, consists essentially of, or consists of silicone oil having a viscosity of at least 10 centistokes, e.g., between 10 and 200,000 centistokes. In various embodiments, the viscosity is between 500 and 10,000 centistokes. At such viscosities, the fluid may be pumped and is not likely to diffuse from the reservoir due to the high molecular weight of the molecules. (As known in the art, higher molecular weight silicone-based or filling fluids typically have a higher viscosity, which is the case for silicone oil.) The nominal fill of the implantable device 125 may be set by measuring one or more of the following parameters: lens capsule geometry, lens size, lens position in the eye, age of the patient, lens estimated refractive index, corneal shape, optical length, along with device characteristics such as power as a function of fill level, estimated fit between lens and the patient capsule, estimated location of lens inside eye, aberrations, lens shape, Zernike polynomials, and/or an equivalent method of determining optical aberrations in the eye. As the implantable device 125 is filled, some or all of these parameters may be monitored, and the nominal fill level may even be adjusted over time.

Sensors and/or other monitoring devices may be utilized to monitor the fill state of the implantable reservoir 125 continuously during filling, or measurements may be taken prior to and/or after one or more fill cycles to determine the fill level with respect to the desired nominal fill level. Filling of the reservoir may occur at fairly high pressures and therefore flow rates. The maximum vacuum pressure attainable to remove fluid at sea level is 1 atmosphere (i.e., full vacuum). During inflation of the reservoir, many atmospheres of pressure may be utilized. The pressure may be determined and/or limited by the pump size and/or maximum pressure that the lines, fittings, and sensors may withstand without failing. Therefore, in various embodiments, the reservoir 125 is slightly underfilled, the fill state is determined, and then another fill cycle is performed to fill the reservoir to the desired nominal level. Such embodiments allow any necessary corrections to be made and may reduce total fill error.

As an example of this technique, for an implanted IOL, the predicted required power of the lens may be 20 diopters based on the patient's corneal aberrometry, expected location of the lens in the eye, and the eye's axial length. According to the patient's capsule size, and the fill properties of the chosen lens, a specific fill volume is determined by the processing module. One may also assume that filling error in the fill system is a percentage of total power that is added (20 diopters in this example). For example, a 5% error would correspond to 1 diopter of power error. Thus, the lens may be initially filled to a level corresponding to 19 diopters to ensure there is no chance of overfill. The refractive state of the eye after this filling is monitored, with expected results between 19 and 20 diopters of power. Based on this measurement, a second volume to be added to the lens is calculated based on measurements (e.g., wavefront aberrometry, pressure inside the lens, optical imaging, and/or ultrasonic imaging of the lens) or a recalculation. If the actual power after the initial fill is 19.5 diopters, then the expected error would be 5% of 0.5 diopter (0.025 diopter). This stepwise approach of filling the lens may continue until the nominal fill level is reached. For the filling of a fluid-filled IOL, monitoring techniques may include but are not limited to wavefront aberrometry, interior pressure measurements, optical imaging, and/or ultrasonic imaging. In addition, filling may be accomplished by monitoring and adjusting aberration and/or Zernike polynomials for optimal depth of field and accommodation as well as refractive state of the lens.

In addition, embodiments of the invention lessen or substantially eliminate the possibility that overfill will decrease the ultimate accuracy of the fill. In the case of an overfill, not only does it take much longer to aspirate (as described above) but the monitoring of fluid being aspirated may become more difficult due to cavitation of air bubbles (per the Boyle's law relationship detailed above). For example, in state 1 the fluid is inside the implantable reservoir 125 near or slightly above atmospheric pressure at a small volume of, for example, 1 μl. When vacuum is applied to aspirate fluid from the implantable reservoir 125, the pressure drop may reach, for example, 10⁻⁴ atmosphere of pressure. This “state 2” results in the initial 1 μl air bubble attaining a volume 10,000 μl, i.e., 10 ml. This relationship would hold true for each air bubble being aspirated. The high-viscosity fluid does not undergo this change in volume since it is effectively incompressible. The large air cavitations resulting from the volumetric change may result in the accuracy of metering and measuring the fluid being aspirated being much less than that during filling. That is, volumetric accuracy during aspiration (i.e., fluid removal) will tend to be lower because of the volume attributable to the air bubbles, making selection of a desired fluid volume more difficult. For example, when infusing air, the air bubble described above would have a maximum compression of only approximately 1 μl, at which point there is no volume of air left to compress. Therefore, knowledge and monitoring of the bubble size and pressure enable the determination of the amount of filling that has occurred—i.e., the net amount of filling liquid transferred. For example, a sensor such as an optical detector may be used to detect the frequency and size of bubbles remaining and/or passing through a fluid line (and/or within a reservoir). Such information may be factored into filling volume calculations as an air bubble compliance adjustment (e.g., subtracted from a total volume of fluid and air transferred using the fluid line).

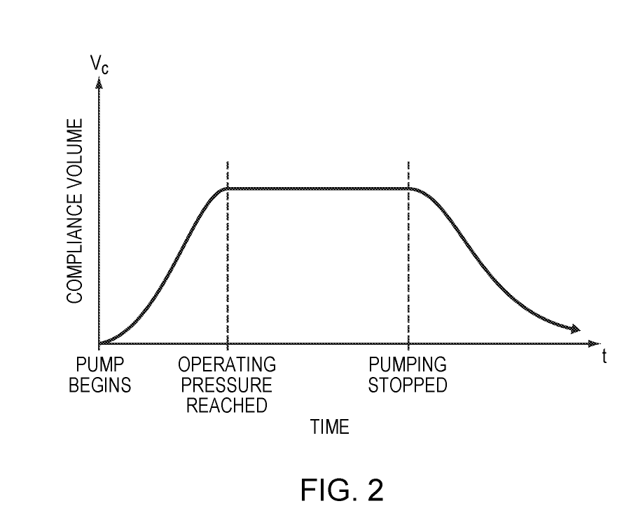

In various embodiments, the high pressure utilized within the system dissipates as the fluid moves through the system. In such embodiments, the highest pressure is typically at the pump, and the pressure decreases as the fluid flows down the line. Thus, when entering the reservoir, the pressure may be low (e.g., less than 0.1 psi). The pressure loss may be due, at least in part, to head loss as the fluid travels down the line. In general, the pressures mentioned herein nominally refer to the highest pressure present in the system (most often the pressure at the pump). FIG. 2 is a graph the half-life relationship of compliance volume (i.e., the additional effective volume due to compliance within the system—e.g., within expanding fluid lines), V_{c}, as a function of time. The graph assumes that at a time of zero the pump begins to pump, and, after some time passes, the operating pressure is attained. This operating pressure is typically dependent on the flow rate along with other factors detailed above. Thus, as the flow rate increases, the operating pressure typically increases as well. As the operating pressure increases, the compliance in the system causes the lines and other expandable elements to increase in volume, resulting in the accumulation of pumped fluid in the system as well as in the reservoir. Thus, in various embodiments of the invention, a non-uniform filling profile is used to minimize the effect of compliance on filling accuracy.

A non-uniform pumping profile may be obtained in a variety of ways but typically accounts for the fact that, after pumping has stopped, the compliance volume decays in the half-life manner shown in FIG. 2. In accordance with embodiments of the invention, non-uniform pumping profiles may include filling with an initial high flow rate and then decreasing the flow rate as the nominal fill of the implantable reservoir is reached in order to reduce the effect of compliance, and/or pausing during the filling procedure for a certain amount of time (which may be expressed as a number of half-life intervals) to allow for the compliance volume to decrease to an acceptable tolerance. In other embodiments, the filling error may be calculated based on wait time, and the filling tip is removed from the reservoir at a specific time after the pump stops (e.g., to allow for the pressure of the system to decrease and for residual fluid to leave the lines), or vacuum is applied at a specific time to remove any additional volume due to compliance and accurately fill the implantable reservoir.

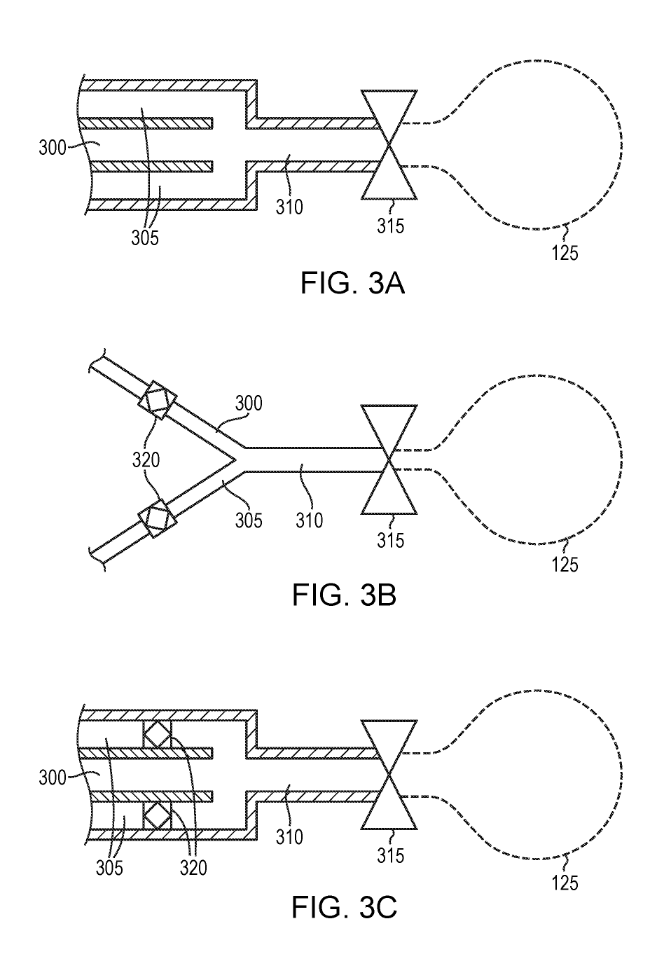

FIGS. 3A-3C depict different configurations, in accordance with embodiments of the invention, in which dead space in the filling system may be minimized. As shown, at least fluid lines 300, 305 may be concentric and join fluidically at a junction with a filling line 310 connected to the implantable reservoir 125. A valve 315 may be present along the fluid line 310, or within implantable reservoir 125, or may not be present at all. Semipermeable membranes 320 may also be placed along one or both of the fluid lines 300, 305. The semipermeable membranes 320 allow air to pass but prevent the filling fluid from passing through. The semipermeable membranes 320 may include, consist essentially of, or consist of a variety of different gas-permeable materials, e.g., polymeric materials such as polyethylene, polytetrafluoroethylene, polyvinylchloride, natural rubber, silicone, or dimethylsilicon rubber. For example, a semipermeable membrane 320 may be the membrane portion of a SEPAREL degassification module available from DIC Corporation of Tokyo, Japan.

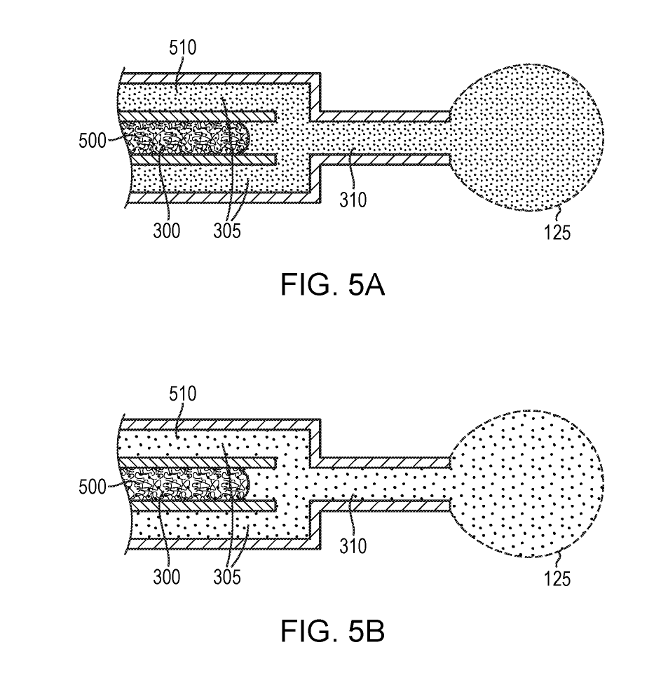

In accordance with embodiments of the invention, dead space is removed from the system via application of vacuum to the fluid line 305 while filling fluid flows through the other fluid line 300. Via the ideal gas law presented above, even if the dead volume remains in the system, the drop in pressure caused by the vacuum removes air (i.e., mass), as also described in more detail below with respect to FIG. 5. The vacuum applied to fluid line 305 evacuates air from any potential dead space within the fluid line 310 not occupied by the filling fluid. The implantable reservoir 125 may also be evacuated by the vacuum. Valve 315 may be closed or opened to control whether or not the implantable reservoir 125 is evacuated. The valve 315 may also be supplemented with or replaced by a plug or another sealing mechanism that seals the fluid line 310. The applied vacuum also draws out any diffused air within the filling fluid. The filling fluid continues to be primed and de-gassed through the line 300.

In the configuration depicted in FIG. 3A, no semipermeable membranes or intermediate valves are present. This configuration may be utilized in a variety of different filling techniques. In one exemplary embodiment utilizing the configuration of FIG. 3A, the filling fluid flows through fluid line 300 until it reaches a specific mark (i.e., volume) in fluid line 300 and/or fluid line 305 prior to reaching the reservoir 125. As the implantable reservoir 125 is filled, the filling fluid will naturally move along fluid line 305; such fluid may be considered to be “unused” fluid that is pumped from the pump but does not enter the implantable reservoir (i.e., fluid that resides in fluid line 305 rather than entering reservoir 125). Knowledge of the initial amount of filling fluid within line 300 and/or line 305 and monitoring of the increasing amount of fluid moving within line 305 may be utilized to determine the amount of unused filling fluid in the system. That is, knowledge of the amount of “unused” filling fluid, as well as the amount actually pumped by the pump, facilitates computation of the amount of filling fluid actually entering the implantable reservoir (i.e., the difference between the total amount of pumped fluid and the amount of unused fluid). Additional filling fluid may be pumped into the reservoir 125 to compensate for this unused filling fluid, ensuring accurate filling of the reservoir 125. In another embodiment, the filling fluid is fully primed though fluid line 305, and then fluid line 305 is closed off. Thereafter, the implantable reservoir 125 is filled.

In the configurations depicted in FIGS. 3B and 3C, a semipermeable membrane 320 is placed in fluid line 305. As the filling fluid moves through fluid line 300, some of the fluid flows into line 305. The filling fluid is primed (i.e., pumped to substantially fill one or more fluid lines prior to introduction of filling fluid into the implantable reservoir) until it reaches the semipermeable membrane 320, through which it may not pass (but air or other gasses can). The filling fluid is then fully primed and may be pumped into the implantable reservoir 125.

Since vacuum is applied to prime the system when the filling fluid is pumped into the implantable reservoir 125, the dead space in the system is diminished proportionally to the amount of vacuum pulled. Even though the filling line 310 may have an initial dead volume of Vᵢ, when the filling fluid is pumped through and the air in this dead volume is forced into the implantable reservoir 125, the volume occupied by air V_{f} is less than Vᵢ (and may be approximately zero depending on the level of vacuum applied) since the pressure inside the reservoir is higher than the initial vacuum used to prime the system). Embodiments of the invention also, in the same manner, remove dead space from the implantable reservoir 125 before introducing fluid therein.

The configurations depicted in FIGS. 3A-3C may include various other features. For example, fluid line 305 may have another opening to allow the implantable reservoir 125 to be purged therethrough. One or more sensors may be placed adjacent to or separate from the permeable membranes 320, and/or on either fluid line 300, 305, to measure the amount of fluid being infused and/or aspirated or to determine when the system is fully purged by determining that no air remains in the implantable reservoir 125. This metering may be accomplished directly with flow sensors or indirectly with, for example, pressure sensors, strain gauges, and/or velocimeters. Such sensors may also be placed within the filling line 310 and measure flow in one or both directions.

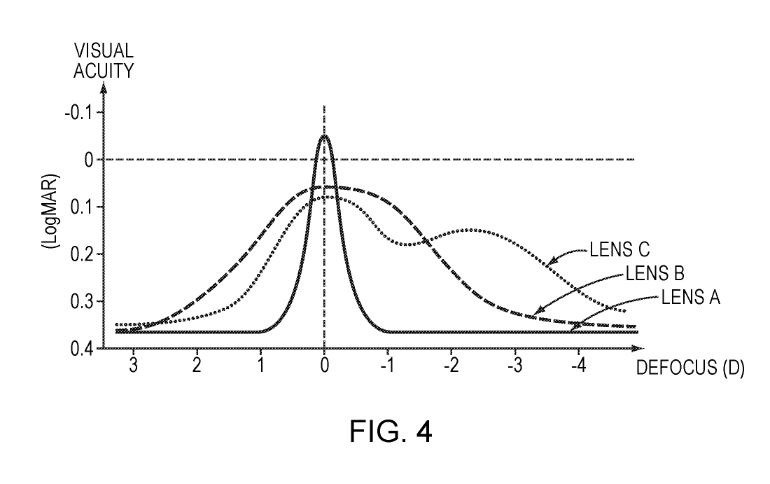

As mentioned herein, many parameters may be monitored and used as an input while fluid filling occurs in conjunction with IOLs. For example, a patient may select certain optical properties and vision parameters before he or she receives the implant. One such property relates to a defocus curve. FIG. 4 depicts exemplary defocus curves for three different lenses A, B, C. A defocus curve shows how well a patient can see (i.e., visual acuity) over a given range of lens powers (i.e., defocus, in diopters). A patient or healthcare practitioner may select a lens based on the defocus curves associated with different lenses and particular optical parameters subject to correction or important to the patient, e.g., field of vision, detail of vision (clarity), high-acuity distance vision, high-acuity near vision, accommodation level, depth of field, astigmatic correction, and/or aberrations. For example, as depicted in FIG. 4, Lens A has the best vision at distance since it has the highest value at zero on the defocus axis (x-axis) but lacks depth of field since the visual acuity decreases rapidly in both directions away from zero on the defocus axis. In contrast, Lens B has a better depth of field while Lens C provides focusing power at two different distances (one far distance, one near distance). Defocus curves may be predicted by monitoring patient parameters and lens characteristics. For example, the fluid properties of a high-viscosity fluid are generally known. As the IOL is filled, the lens shape in the patient's eye may be monitored using one or more methods including but not limited to wavefront aberrometry, interior pressure monitoring (whereby the lens shape is inferred based on the pressure), optical imaging, and/or ultrasonic imaging of the lens. Such information along with the lens and fluid properties may predict a patient's vision after implantation and filling—e.g., the defocus behavior shown in FIG. 4. Such predictions of lens behavior and vision response may be matched to the preferred vision characteristics desired by the patient. In addition, if the IOL includes more than one reservoir, desired aberrations may be used to adjust the amount of filling in one or more of the reservoirs. For example, to correct astigmatism, one fill reservoir of the IOL may be filled (or prefilled) to 30 microliters, while another may be filled to 100 microliters, resulting in local alterations of the overall shape of the IOL.

FIGS. 5A and 5B depict two different states experienced by the system illustrated in FIGS. 3A-3C when dead space is evacuated as described above. FIG. 5A shows a filling fluid 500 in the fluid line 300 with air 510 at atmospheric pressure (depicted as the dotted area) occupying space in the evacuation line 305, filling line 310, and the implantable reservoir 125. FIG. 5B depicts the system as a vacuum is pulled and the pressure drops. There is less air 510 occupying the dead space (as indicated by the sparser dots), and therefore when the fluid 500 in the fluid line 300 is injected into the implantable reservoir 125, less air enters the implantable reservoir 125. The vacuum pulled through fluid line 305 may also be used to draw the fluid from either a reservoir upstream from the fluid line 300 or to draw the fluid at the very end of priming (i.e., when the fluid line 300 is substantially filled with filling fluid) to the filling line 310. This minimizes the amount of fluid waste and allows for accurate priming of the reservoir 125 attached to the fluid line 310 prior to priming. The vacuum may also apply a negative pressure at the meniscus of the fluid, which may extract dissolved and/or dispersed gas (e.g., air) from the fluid 500.

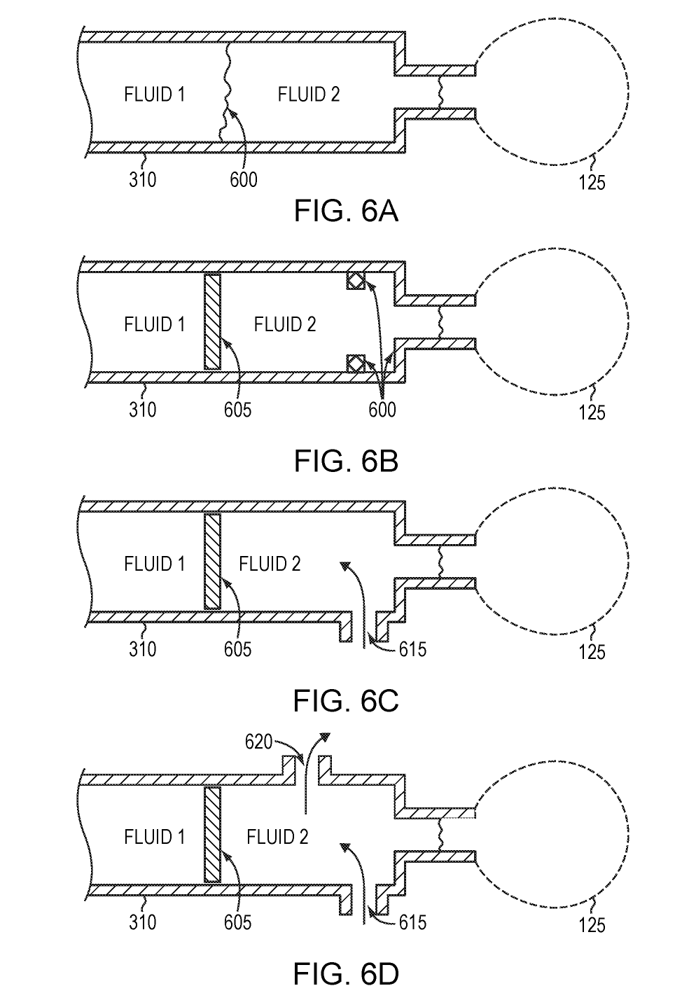

In various embodiments of the present invention, a different fluid (e.g., a different liquid) may be utilized to force the filling fluid into the inflatable reservoir, as depicted in FIGS. 6A-6D. Such embodiments may be advantageously deployed, for example, in the case of rare and/or expensive filling fluids, as loss or non-utilization of the filling fluid is minimized. In addition, such embodiments may be employed to accurately fill by prefilling the fluid lines with a specific volume of filling fluid and then pumping only that amount of fluid into the reservoir. Thus, the predetermined volume of filling fluid is pushed (or pulled) into the fluid line(s), and then another fluid is forced into the line(s) behind the filling. This allows for most of the priming to be done with the pushing fluid (herein referred to and illustrated as “fluid 1”) while the filling fluid (herein also referred to as “fluid 2”) is pushed (or pulled) through a fluidic line towards and into the implantable reservoir ahead of fluid 1; in this way, all of the filling fluid but none of fluid 1 enters the implantable reservoir. In the configuration depicted in FIG. 6A, fluid 1 and fluid 2 are in contact with each other but are immiscible and thus form an interface or boundary 600 between them. The interface 600 may also include or consist essentially of a trapped air pocket between fluid 1 and fluid 2. FIG. 6B depicts a configuration in which a physical (solid) barrier or membrane 605 is disposed between fluid 1 and fluid 2. In this configuration, fluid 1 may push the barrier 605 and, thus, fluid 2 toward the reservoir 125 while minimizing or substantially eliminating the chances of mixing between fluids 1 and 2. As shown in FIG. 6B, the fluid line 310 may include one or more stops 610 that arrest movement of the barrier 605, thereby halting the flow of fluid 2 into the reservoir 125.

FIG. 6C depicts a configuration demonstrating a technique of priming fluid 2 downstream of fluid 1. In the configurations depicted in FIGS. 6A and 6B, fluid 2 and fluid 1 may have been introduced at the beginning of the fluidic line 310, or the line 310 may have been pre-primed with fluid 2 in front of fluid 1, or fluids 1 and 2 may have entered a common line from different reservoirs. In contrast, FIG. 6C depicts a configuration in which fluid 2 is pumped (or otherwise introduced) into the fluidic line 310 through an opening 615 in the fluidic line. After priming (i.e., filling fluid line 310 with the desired amount of fluid 2), opening 615 is typically closed so that fluid 1 can push fluid 2 into the implantable reservoir 125. As shown, the barrier or membrane 605 may be used in this configuration to prevent fluid intermixing. FIG. 6D depicts a configuration similar to that depicted in FIG. 6C. In this configuration of FIG. 6D, fluid 2 is again primed through opening 615 but a relief opening 620 in the fluidic line 310 allows for air to escape or a vacuum to be pulled to reduce the dead space, as detailed herein.

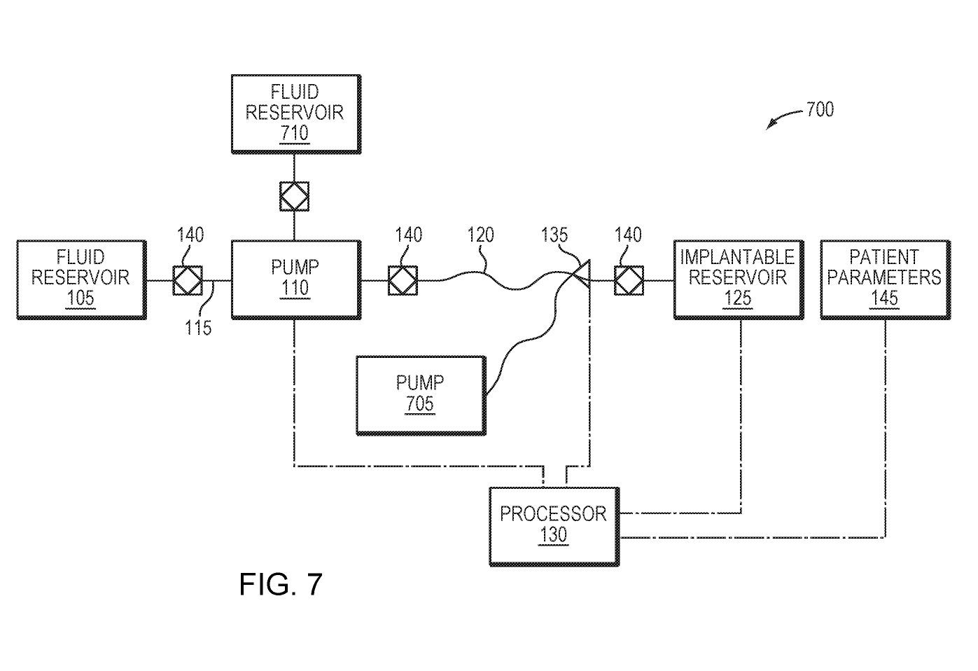

FIG. 7 schematically illustrates another exemplary filling and aspiration system 700 for a high-viscosity fluid in accordance with embodiments of the present invention. As shown, system 700 includes not only pump 110 but also a pump 705 utilized to pull vacuum, eliminate dead space within the fluid lines, and assist in priming the fluid within fluid line 120. In this manner, as detailed herein, diffused air within the fluid will be evacuated from the system during priming of the fluid. For example, with reference to FIG. 6D, pump 110 primes and pumps fluid through the fluid line 310 and a vacuum may be pulled through the opening 620.

System 700 also includes not only fluid reservoir 105 but also fluid reservoir 710. Fluid reservoir 710 may contain, for example, non-filling fluid (i.e., a liquid) utilized to push the filling fluid through the lines and into implantable reservoir 125. Embodiments of the invention may include more than two pumps and/or more than two fluid reservoirs. For example, another pump may be disposed at the end of the fluid line 120 near the implantable reservoir 125, in order to allow for, e.g., another vacuum line or for the priming of fluid to the front of the fluid line 120. As detailed above for system 100, one or more sensors 135 may be disposed at different points on the fluid line 120. Sensors may even be placed at or near the pumps 110, 705 and fluid reservoirs 105, 710. Signals from the sensors may be utilized by the control system 130 to control and regulate the filling and evacuation of the system 700.

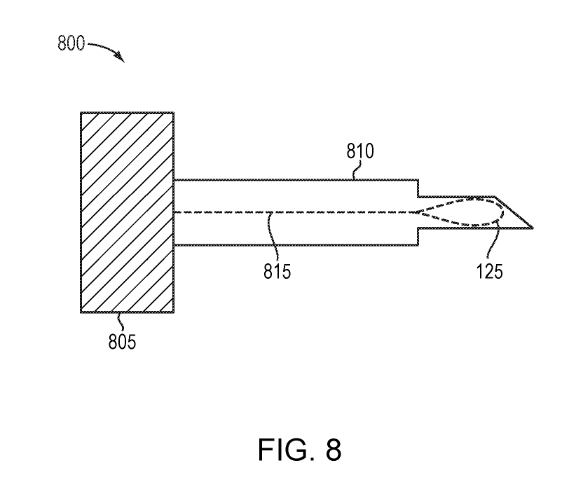

Filling systems in accordance with embodiments of the present invention may feature fluid reservoirs and pumps integrated into single units. For example a syringe or similar instrument may feature both a fluid reservoir and a pump, as it both contains and administers filling fluid. In such embodiments, the fluid line for fluid coupling to the implantable reservoir may include, consist essentially of, or consist of, for example, tubing, concentric tubing, and/or a needle. FIG. 8 depicts an exemplary handheld system 800 for high-viscosity filling in accordance with embodiments of the present invention. As shown, system 800 features a fluid reservoir 805 that may be provided prefilled with a filling fluid (e.g., a pharmaceutical agent). The fluid reservoir 805 is coupled to a hand piece 810 that contains a fluidic line 815. Fluidic line 815 is utilized to fluidly couple fluid reservoir 805 with an implantable reservoir 125. In various embodiments of the invention, system 800 features multiple fluid lines, fluid-line configurations, and/or sensors for fill monitoring as detailed herein. As shown, the implantable reservoir 125 may initially be in a deflated state and disposed within a portion of hand piece 810 (e.g., the distal end, as shown) for implantation into a patient (e.g., into the patient's eye) and filling. In embodiments in which the implantable reservoir 125 includes or consists essentially of an IOL, different fluid reservoirs 805 may be available with different pre-set amounts of the filling fluid therewithin; these pre-set amounts of fluid may approximately correspond to particular desired base powers or other characteristics of the IOL (e.g., power of lens, accommodation (both amplitude and sensitivity), and/or toric curvature). In various embodiments, the fluid reservoir 805 may instead or in addition have markers disposed therein or thereon indicating different fluid volumes (and/or correlation to base power or another desired reservoir characteristic). Such markers may include or consist essentially of, e.g., adjustable stops for a plunger system (e.g., c-collars), pipettes, and/or controlled motor priming system. (In a controlled motor priming system, a motor or pressurized source may be controlled to prime the system to a correct fill point. Once the fill point is reached, the controlled motor may either be detached or remain in connection with the fluid reservoir 805 before filling begins. In various embodiments, sensors in the fluidic line 815 may be used as a feedback loop to control the motor.)

In various embodiments, the fluid reservoir 805 may be pre-assembled with the hand piece 810 as a single unit, or the fluid reservoir 805 may be detachable from the hand piece 810. In detachable embodiments, multiple different fluid reservoirs 805 may be utilized as described herein. For example, a first fluid reservoir 805 may contain the fluid to fill the implantable reservoir 125. That fluid reservoir 805 may then be detached, and a second fluid reservoir 805 (containing a different fluid) may be attached and utilized to deliver (i.e., push) the first fluid into the implantable reservoir 125. The system 800 may include components of, and/or operate in accordance with, refill systems detailed in U.S. patent application Ser. No. 14/579,231, filed on Dec. 22, 2014, the entire disclosure of which is incorporated by reference herein. For example, the hand piece may be ergonomically shaped for handling by the clinician refilling the implantable reservoir, may incorporate one or more external switches or actuators, control circuitry and/or one or more pumps for pumping fluid or applying vacuum, and/or may terminate in a refill needle (or other fluid line) that interfaces with the implantable reservoir (e.g., via a valve thereon).

The fluid reservoir 805 may utilize any suitable mechanism to dispense high-viscosity fluid in accordance with embodiments of the invention. In various embodiments, the fluid reservoir 805 is pre-pressurized at a pressure higher than the internal pressure of the implantable reservoir 125, enabling flow of the high-viscosity fluid into the implantable reservoir 125. In other embodiments, tubes or other conduits are fluidly coupled to the fluid reservoir 805, fluid line 815, and/or hand piece 810 that may be pressurized or utilized to pull vacuum. A pressurized line may exert force directly onto the high-viscosity fluid for delivery or push a plunger or membrane as shown in FIGS. 6B-6D. Vacuum and pressurized lines may also be cooperatively utilized to de-gas, prime, and calibrate for correct fill as described herein. Other embodiments for fluid delivery include the use of electrolysis, springs, hydraulics, or manual actuation. In various embodiments, the use of hydraulic advantage may be used to overcome the high pressure needed to deliver the high-viscosity fluid. For example, a longer delivery stroke and/or the minimization of the contact area of the delivery fluid onto the plunger may be utilized to provide hydraulic advantage and thus enable use of smaller amounts of force to apply larger pressures to high-viscosity fluids.

The implantable reservoirs may each contain one or more valves accessible externally with a needle or other fluid line for filling. Such valves may be self-sealing, e.g., as described in U.S. patent application Ser. No. 14/980,116, filed on Dec. 28, 2015, the entire disclosure of which is incorporated by reference herein. The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for filling an implantable reservoir with a high-viscosity fluid, the system comprising:
a first fluid line for fluidly coupling to the implantable reservoir;
fluidly coupled to the first fluid line, a second fluid line for conducting the high-viscosity fluid to the first fluid line;
fluidly coupled to the first and second fluid lines, a first pump for evacuating air from at least the first fluid line, thereby eliminating dead space therefrom;
fluidly coupled to the second fluid line, a first fluid reservoir for containing the high-viscosity fluid; and
a second pump for pumping high-viscosity fluid from the first fluid reservoir to the implantable reservoir via the first fluid line.

2. The system of claim 1, wherein the first and second fluid lines are concentric, the second fluid line terminating within the first fluid line upstream of a terminus thereof, the terminus of the first fluid line being coupled to the implantable reservoir.

3. The system of claim 1, further comprising a control system for controlling at least one of the first or second pumps.

4. The system of claim 3, further comprising one or more sensors for measuring flow rate and/or pressure within at least one of the first or second fluid lines, the control system being responsive to signals received from the one or more sensors.

5. The system of claim 3, wherein (i) the implantable reservoir comprises an intraocular lens, and (ii) the control system controls the flow of the high-viscosity fluid into the implantable reservoir based on one or more patient parameters.

6. The system of claim 5, wherein the one or more patient parameters comprise one or more of lens capsule geometry, lens size, lens position in the eye, patient age, corneal shape, lens refractive index, desired optical power, accommodation, or nominal fill of the intraocular lens.

7. The system of claim 1, further comprising a valve configured to fluidly couple the first fluid line to the implantable reservoir and fluidly uncouple the first fluid line from the implantable reservoir.

8. The system of claim 1, further comprising one or more semipermeable membranes disposed within the second fluid line, the one or more semipermeable membranes allowing flow of gas therethrough without allowing flow of liquid therethrough.

9. The system of claim 1, further comprising, fluidly coupled to the second fluid line, a second fluid reservoir for containing a pushing fluid for exerting force on the high-viscosity fluid.

10. The system of claim 1, wherein the system is a handheld system.

11. A method of filing an implantable reservoir with high-viscosity fluid though a first fluid line fluidly coupled to the implantable reservoir, the method comprising:
introducing the high-viscosity fluid into a second fluid line fluidly coupled to the first fluid line;
evacuating air from the first fluid line; and
thereafter, urging the high-viscosity fluid into the implantable reservoir via the first fluid line.

12. The method of claim 11, wherein the high-viscosity fluid is introduced into the second fluid line via a third fluid line fluidly coupled to the first and second fluid lines.

13. The method of claim 11, further comprising, before urging the high-viscosity fluid into the implantable reservoir, evacuating air from the implantable reservoir via the first fluid line.

14. The method of claim 11, wherein the first and second fluid lines are concentric, the second fluid line terminating within the first fluid line upstream of a terminus thereof, the terminus of the first fluid line being coupled to the implantable reservoir.

15. The method of claim 11, wherein the high-viscosity fluid is urged into the implantable reservoir by a pushing fluid disposed within the second fluid line.

16. The method of claim 11, further comprising evacuating air from at least a portion of the second fluid line prior to introducing the high-viscosity fluid into the second fluid line.

17. A method of filing an implantable reservoir with a pre-determined amount of high-viscosity fluid though a fluid line fluidly coupled to the implantable reservoir, the method comprising:
disposing the pre-determined amount of high-viscosity fluid into a first region of the fluid line;
disposing a pushing fluid into a second region of the fluid line, wherein (i) the pushing fluid is different from the high-viscosity fluid and (ii) the first region of the fluid line is disposed upstream of the implantable reservoir and downstream of the second region of the fluid line; and
applying force to the pushing fluid to thereby urge the high-viscosity fluid into the implantable reservoir, substantially no pushing fluid entering the implantable reservoir.

18. The method of claim 17, wherein the high-viscosity fluid and the pushing fluid are immiscible and in contact with each other within the fluid line.

19. The method of claim 17, wherein a moveable mechanical boundary is disposed between the pushing fluid and the high-viscosity fluid, movement of the mechanical boundary urging the high-viscosity fluid into the implantable reservoir.

20. The method of claim 17, further comprising evacuating air from the first region of the fluid line before disposing the high-viscosity fluid therein.
